(19) United States
(12) Patent Application Publication
(10) Pub. No.: US 2025/0295332 A1
(43) Pub. Date: Sep. 25, 2025

(54) PHOTOACOUSTICS FOR NON-INVASIVE GLUCOSE SENSING

(71) Applicant: QUALCOMM Incorporated, San Diego, CA (US)

(72) Inventors: Emily Kathryn BROOKS, Amherst, NY (US); John Keith SCHNEIDER, Williamsville, NY (US); Ye ZHAN, Buffalo, NY (US); Evan Michael BRELOFF, Kenmore, NY (US); Hrishikesh Vijaykumar PANCHAWAGH, Cupertino, CA (US); Nicholas BUCHAN, San Jose, CA (US)

(21) Appl. No.: 18/610,118

(22) Filed: Mar. 19, 2024

(51) Int. Cl. A61B 5/145; A61B 5/00

(52) U.S. Cl. CPC A61B 5/14532; A61B 5/0095; A61B 5/7246; A61B 5/7278

(57) ABSTRACT

Disclosed are systems and techniques for glucose sensing. For example, an example of a process can include transmitting, by at least one transmitter into living tissue, a first signal with a first wavelength, a second signal with a second wavelength, and a third signal with a third wavelength to produce an acoustic response from the living tissue. The first wavelength has a first correlation with the blood glucose concentration, the second wavelength has a second correlation with the blood glucose concentration, and the third wavelength has a third correlation with the blood glucose concentration. The process can include receiving, by at least one receiver, a response signal of the acoustic response. The process can include determining, by at least one processor, the blood glucose concentration based on a photoacoustic spectrum of the response signal.

### FIELD

The present disclosure generally relates to glucose sensing. For example, aspects of the present disclosure relate to utilizing photoacoustics for non-invasive glucose sensing.

### BACKGROUND

The prevalence of diabetes is increasing globally. Continuous blood glucose monitoring is essential to control the disease and avoid long-term complications. Diabetics suffer on a daily basis with the traditional glucose monitors currently in use, which are invasive, painful, and cost-intensive. Therefore, the demand for non-invasive, painless, economical, and reliable approaches to monitor glucose levels is increasing. Researchers and scientists have been working on the enhancement of these technologies to achieve better results. Many different glucose sensing technologies have been developed. Some methods are non-invasive, but are not able to effectively isolate on an artery or vein to measure the blood glucose concentration. As such, there is a need for improved systems and techniques for non-invasive monitoring (e.g., sensing) of blood glucose concentration.

### SUMMARY

The following presents a simplified summary relating to one or more aspects disclosed herein. Thus, the following summary should not be considered an extensive overview relating to all contemplated aspects, nor should the following summary be considered to identify key or critical elements relating to all contemplated aspects or to delineate the scope associated with any particular aspect. Accordingly, the following summary has the sole purpose to present certain concepts relating to one or more aspects relating to the mechanisms disclosed herein in a simplified form to precede the detailed description presented below.

Disclosed are systems and techniques for utilizing photoacoustics for non-invasive glucose sensing. According to at least one example, an apparatus configured to determine a blood glucose concentration is provided. The apparatus includes: at least one transmitter configured to transmit, into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; at least one receiver configured to receive a response signal of the acoustic response; and at least one processor configured to determine the blood glucose concentration based on a photoacoustic spectrum of the response signal

In another illustrative example, a method is provided for determining a blood glucose concentration. The method includes: transmitting, by at least one transmitter into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; receiving, by at least one receiver, a response signal of the acoustic response; and determining, by at least one processor, the blood glucose concentration based on a photoacoustic spectrum of the response signal.

In another illustrative example, a non-transitory computer-readable medium is provided having stored thereon instructions that, when executed by one or more processors, cause the one or more processors to: cause at least one transmitter to transmit, into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; cause at least one receiver to receive a response signal of the acoustic response; and determine the blood glucose concentration based on a photoacoustic spectrum of the response signal.

In another illustrative example, an apparatus configured to determine a blood glucose concentration is provided. The apparatus includes: means for transmitting, into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; means for receiving a response signal of the acoustic response; and means for determining the blood glucose concentration based on a photoacoustic spectrum of the response signal.

Aspects generally include a method, apparatus, system, computer program product, non-transitory computer-readable medium, user device, user equipment, wireless communication device, and/or processing system as substantially described with reference to and as illustrated by the drawings and specification.

In some aspects, each of the apparatuses described herein is, can be part of, or can include a mobile device, a smart or connected device, a camera system, and/or an extended reality (XR) device (e.g., a virtual reality (VR) device, an augmented reality (AR) device, or a mixed reality (MR) device). In some examples, the apparatuses can include or be part of a wearable device (e.g., a watch, a ring, or other wearable device), a mobile device (e.g., a mobile telephone or so-called “smart phone” or other mobile device), a personal computer, a laptop computer, a tablet computer, a server computer, a robotics device or system, a vehicle, an aviation system, or other device. In some aspects, each apparatus can include an image sensor (e.g., a camera) or multiple image sensors (e.g., multiple cameras) for capturing one or more images. In some aspects, each apparatus can include one or more displays for displaying one or more images, notifications, and/or other displayable data. In some aspects, each apparatus can include one or more speakers, one or more light-emitting devices, and/or one or more microphones. In some aspects, each apparatus can include one or more sensors. In some cases, the one or more sensors can be used for determining a location of the apparatuses, a state of the apparatuses (e.g., a tracking state, an operating state, a temperature, a humidity level, and/or other state), a state of a person wearing the apparatus (e.g., a blood glucose level or blood glucose concentration of the person, etc.), and/or for other purposes.

Some aspects include a device having a processor configured to perform one or more operations of any of the methods summarized above. Further aspects include processing devices for use in a device configured with processor-executable instructions to perform operations of any of the methods summarized above. Further aspects include a non-transitory processor-readable storage medium having stored thereon processor-executable instructions configured to cause a processor of a device to perform operations of any of the methods summarized above. Further aspects include a device having means for performing functions of any of the methods summarized above.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims. The foregoing, together with other features and aspects, will become more apparent upon referring to the following specification, claims, and accompanying drawings.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The preceding, together with other features and embodiments, will become more apparent upon referring to the following specification, claims, and accompanying drawings.

### BRIEF DESCRIPTION OF THE DRAWINGS

Illustrative aspects of the present application are described in detail below with reference to the following figures:

FIG. 1 is a diagram illustrating example components of a device, in accordance with some examples.

FIG. 2 is a diagram illustrating an example process utilizing photoacoustics for non-invasive glucose sensing, in accordance with some examples.

FIG. 3 is a diagram illustrating an example of depth discrimination by using photoacoustics, in accordance with some examples.

FIG. 4 is a table illustrating different ratios of photoacoustic intensities of a response signal with a photoacoustic spectrum based on different wavelengths, where the ratios may be employed when utilizing photoacoustics for non-invasive glucose sensing, in accordance with some examples.

FIG. 5 is a graph illustrating an example of a photoacoustic spectra of various different glucose concentrations, in accordance with some examples.

FIG. 6 is a flow diagram illustrating an example of a process for glucose sensing, in accordance with some examples.

FIG. 7 is a diagram illustrating an example of a system for implementing certain aspects described herein.

### DETAILED DESCRIPTION

Certain aspects of this disclosure are provided below for illustration purposes. Alternate aspects may be devised without departing from the scope of the disclosure. Additionally, well-known elements of the disclosure will not be described in detail or will be omitted so as not to obscure the relevant details of the disclosure. Some of the aspects described herein can be applied independently and some of them may be applied in combination as would be apparent to those of skill in the art. In the following description, for the purposes of explanation, specific details are set forth in order to provide a thorough understanding of aspects of the application. However, it will be apparent that various aspects may be practiced without these specific details. The figures and description are not intended to be restrictive.

The ensuing description provides example aspects only, and is not intended to limit the scope, applicability, or configuration of the disclosure. Rather, the ensuing description of the example aspects will provide those skilled in the art with an enabling description for implementing an example aspect. It should be understood that various changes may be made in the function and arrangement of elements without departing from the scope of the application as set forth in the appended claims.

The terms “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspects of the disclosure” does not require that all aspects of the disclosure include the discussed feature, advantage or mode of operation.

Diabetes mellitus (often referred to simply as diabetes) is a common metabolic disease that can lead to serious complications, such as cardiovascular disease, stroke, blindness, chronic renal failure, neuropathy, and even death. Diabetes is a growing global challenge as one in eleven people presently suffer from diabetes worldwide, which is expected to nearly double within the next ten years. When the blood glucose level (e.g., blood glucose concentration) in a body is uncontrolled, diabetes can lead to complications that can result in a lifetime of consequences for a patient. As such, regular monitoring and subsequent immediate control of the blood glucose level is required.

Some current available methods for monitoring and control of blood glucose levels are based on enzyme reactions that require a painful puncturing procedure of a fingertip with a lance to extract the blood invasively. This procedure can make a patient unwilling to check their glucose level as frequently as required and can have a risk of wound infection. These invasive glucose monitoring methods cannot provide continuous glucose monitoring for control.

Other current available methods for monitoring and control of the blood glucose level are based on determining the glucose level by measuring interstitial fluid glucose (IFG). However, the glucose concentration measurement obtained from IFG has a seven to fifteen minute lag in what glucose level the brain is experiencing and, as such, glucose concentration measurements obtained from IFG can be problematic for developing a closed loop control for an insulin pump. These methods measuring IFG require an invasive breaking of the skin barrier (by microneedles), changing the sensor frequently (often weekly), and calibration by a finger prick of up to twice a day.

To address the challenges of these invasive glucose monitoring methods, numerous efforts have been made towards developing alternative non-invasive methods of glucose detection comparable to the currently available invasive techniques. Some non-invasive methods for monitoring and control of the blood glucose level are based on the use of optical spectroscopy. However, for these optical methods the analysis of the received light signal is inherently complex because the glucose signal is often very weak and easily interferes with other signals from a variety of molecules in the blood and tissue. The received light signal is dominated by glucose and water absorption, and the spectroscopy sensors cannot differentiate between signals caused by glucose absorption or scattering and signals caused by water absorption. These optical methods are also vulnerable to the variability and inhomogeneity of human skin, which continually changes due to normal physiology, and these methods have no ability to isolate on an artery or vein to measure the blood glucose level. The optical methods require frequent calibration and multiple wavelengths must be pulsed nearly simultaneously to capture readings without any background fluctuations. Optical methods may also be limited by penetration depth. As such, improved systems and techniques for non-invasive glucose monitoring can be beneficial.

In one or more aspects, systems, apparatuses, processes (also referred to as methods), and computer-readable media (collectively referred to herein as “systems and techniques”) are described herein that utilize photoacoustics for non-invasive glucose sensing. In one or more aspects, the systems and techniques provide solutions for non-invasive glucose monitoring that employ photoacoustic ratiometric glucose (PARG) sensing that utilizes photoacoustic spectroscopy. The systems and techniques enable wearable, calibration-free, continuous, non-invasive measurements of blood glucose concentration. Blood glucose measurements require a deeper penetration into the tissue than the methods that target IFG measurements to be able to obtain a glucose measurement that is instantaneous and represents what glucose level the brain is presently experiencing. PARG allows for depth discrimination on an artery or vein to enable a direct glucose measurement from a wearable device.

Light absorption is affected by glucose concentration, especially in the near infrared (NIR) region of the electromagnetic spectrum. The NIR region includes wavelengths ranging from 780 nanometers (nm) to 2500 nm. The correlation of glucose concentration with light absorption can be positive, negative, or minimal (e.g., approximately zero), depending upon the wavelength being used. PARG utilizes signals with three different wavelengths to obtain a photoacoustic spectrum to determine blood glucose concentration. In one or more examples, a first wavelength has a positive correlation with the blood glucose concentration, a second wavelength has a negative correlation with the blood glucose concentration, and a third wavelength has a minimal (e.g., approximately zero) correlation with the blood glucose concentration. In some examples, the first wavelength has a first positive correlation with the blood glucose concentration, the second wavelength has a second positive correlation with the blood glucose concentration, and the third wavelength has a minimal (e.g., approximately zero) correlation with the blood glucose concentration, where the first positive correlation and the second positive correlation are different positive correlations. In one or more examples, the first wavelength has a first negative correlation with the blood glucose concentration, the second wavelength has a second negative correlation with the blood glucose concentration, and the third wavelength has a minimal (e.g., approximately zero) correlation with the blood glucose concentration, where the first negative correlation and the second negative correlation are different negative correlations. The use of signals with three different wavelengths allows for the normalization of the glucose measurements as well as for a calibration-free approach. The use of signals with three different wavelengths also allows for an elimination of physiological features (e.g., blood volume change, body temperature, movement, etc.) other than glucose affecting the signals.

In one or more aspects, the systems and techniques described herein for determining blood glucose concentration utilize a photoacoustic spectrum of blood glucose concentration. For example, a photoacoustic system is provided that obtains the photoacoustic spectrum of blood glucose concentration from in vivo living tissue of a patient. In the photoacoustic system, a transmitter (e.g., one or more vertical cavity surface emitting lasers (VCSELs), such as a VCSEL array) can be used to radiate signals towards the living tissue to produce an acoustic response from the living tissue. In one or more examples, the signals are transmitted (e.g., pulsed) sequentially within one or more (e.g., one, two, three, or etc.) microseconds from one another.

In one or more examples, the signals include a first signal with a first wavelength (λ₁), a second signal with a second wavelength (λ₂), and a third signal with a third wavelength (λ₃). In some cases, the first wavelength has a first correlation with the blood glucose concentration, the second wavelength has a second correlation with the blood glucose concentration, and the third wavelength has a third correlation with the blood glucose concentration. In one or more examples, the first correlation is a first positive correlation with the blood glucose concentration and the second correlation is a first negative correlation with the blood glucose concentration, the first correlation is a second positive correlation with the blood glucose concentration and the second correlation is a third positive correlation with the blood glucose concentration, or the first correlation is a second negative correlation with the blood glucose concentration and the second correlation is a third negative correlation with the blood glucose concentration. In some cases, the first, second, and third positive correlations are different positive correlations. In some cases, the first, second, and third negative correlations are different negative correlations.

When the laser beam irradiates the living tissue, a thermal expansion within the tissue is produced, which generates an acoustic response in the form of an acoustic wave. The photoacoustic system can employ a receiver for receiving a response signal (having a photoacoustic spectrum) of the acoustic response (e.g., produced by the acoustic response). At least one processor (e.g., of the photoacoustic system) can determine a ratiometric analysis of the blood glucose concentration C_{gl} based on the received response signal. For instance, the at least one processor can determine a ratio (R) of characteristics of the response signal based on the first wavelength over characteristics of the response signal based on the second wavelength over characteristics of the response signal based on the third wavelength.

In one or more examples, the characteristics may be a photoacoustic intensity, a time integral (e.g., an area under the curve), or signal features (e.g., signal shape). For example, the at least one processor can determine a ratio (R) of a photoacoustic intensity of the response signal based on the first wavelength (e.g., ΔI_{OA}(λ₁)) over a photoacoustic intensity of the response signal based on the second wavelength (e.g., ΔI_{OA}(λ₂)) over a photoacoustic intensity of the response signal based on the third wavelength (e.g., ΔI_{OA}(λ₃)), such that R=(ΔI_{OA}(λ₁)/ΔI_{OA}(λ₂))/ΔI_{OA}(λ₃), where I_{OA}(λ) represents the intensity (I) of optical absorption (OA) at a particular wavelength a. In one or more examples, the blood glucose concentration can be determined based on the determined ratio (R).

In one or more examples, the characteristics of the response signal based on the third wavelength can be a normalization factor. In some examples, the first signal, the second signal, and the third signal may be transmitted within ten microseconds or nanoseconds of each other. In one or more examples, the first wavelength, the second wavelength, and the third wavelength are each a NIR wavelength. In some examples, the blood glucose concentration is an absolute value (e.g., a quantity value). In one or more examples, the third correlation is between the first correlation and the second correlation. In some examples, the third correlation with the blood glucose concentration is approximately zero. In one or more examples, each transmitter of the one or more transmitters is a vertical-cavity surface-emitting laser (VCSEL).

Additional aspects of the present disclosure are described in more detail below.

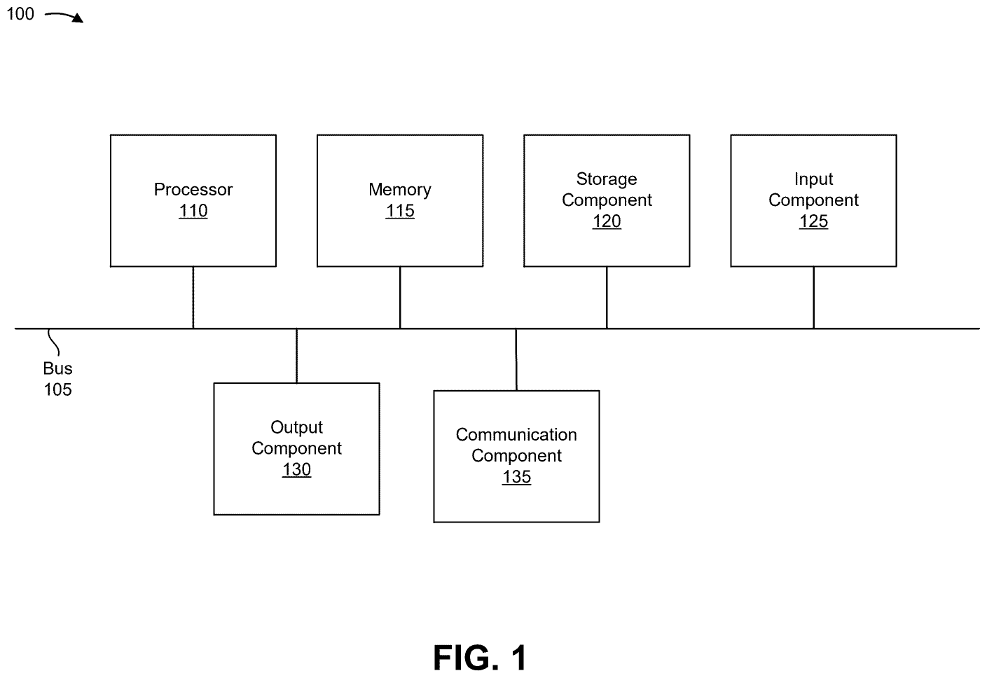

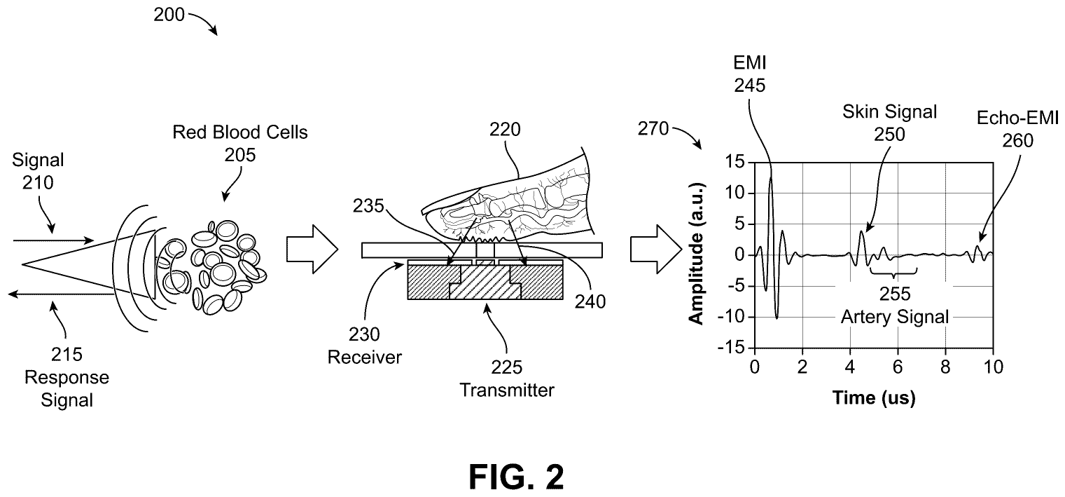

FIG. 1 is a diagram illustrating example components of a device 100, in accordance with the present disclosure. The device 100 can include any type of device configured to measure blood glucose level (e.g., blood glucose concentration) of a subject, such as a person. For instance, the device 100 can include a wearable device (e.g., a watch, a ring, a bracelet, or other type of wearable device). Device 100 may correspond to or include a transmitter (e.g., the transmitter 225 of FIG. 2, which may be a light source), a receiver (e.g., receiver 230 of FIG. 2), and/or a transducer (e.g., which may include the transmitter 225 and the receiver 230 of FIG. 2). For example, the transmitter 225 and the receiver 230 of FIG. 2 may be located within the device 100. In some aspects, the transmitter (e.g., the transmitter 225 of FIG. 2), receiver (e.g., receiver 230 of FIG. 2), and/or transducer (e.g., which may include the transmitter 225 and the receiver 230 of FIG. 2) may include one or more devices 100 and/or one or more components of device 100. As shown in FIG. 1, device 100 may include a bus 105, a processor 110, a memory 115, a storage component 120, an input component 125, an output component 130, and/or a communication component 135.

Bus 105 may include a component that permits communication among the components of device 100. Processor 110 may be implemented in hardware, firmware, or a combination of hardware and software. Processor 110 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some aspects, processor 110 may include one or more processors capable of being programmed to perform a function. Memory 115 may include a random access memory (RAM), a read only memory (ROM), and/or another type of dynamic or static storage device (e.g., a flash memory, a magnetic memory, and/or an optical memory) that stores information and/or instructions for use by processor 110.

Storage component 120 can store information and/or software related to the operation and use of device 100. For example, storage component 120 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 125 may include a component that permits device 100 to receive information, such as via user input (e.g., a touch screen display, a keyboard, a keypad, a mouse, a button, a switch, and/or a microphone). Additionally, or alternatively, input component 125 may include a component for determining a position or a location of device 100 (e.g., a global positioning system (GPS) component or a global navigation satellite system (GNSS) component) and/or a sensor for sensing information (e.g., an accelerometer, a gyroscope, an actuator, or another type of position or environment sensor). Output component 130 can include a component that provides output information from device 100 (e.g., a display, a speaker, a haptic feedback component, and/or an audio or visual indicator).

Communication component 135 may include one or more transceiver-like components (e.g., a transceiver and/or a separate receiver and transmitter) that enables device 100 to communicate with other devices, such as via a wired connection, a wireless connection, or a combination of wired and wireless connections. Communication component 135 may permit device 100 to receive information from another device and/or provide information to another device. For example, communication component 135 may include an Ethernet interface, an optical interface, a coaxial interface, an infrared interface, a radio frequency interface, a universal serial bus (USB) interface, a wireless local area interface (e.g., a Wi-Fi interface or a BLE interface), and/or a cellular network interface.

Communication component 135 may include one or more antennas for receiving wireless radio frequency (RF) signals transmitted from one or more other devices, cloud networks, and/or the like. The antenna may be a single antenna or an antenna array (e.g., antenna phased array) that can facilitate simultaneous transmit and receive functionality. The antenna may be an omnidirectional antenna such that signals can be received from and transmitted in all directions. The wireless signals may be transmitted via a wireless network. The wireless network may be any wireless network, such as a cellular or telecommunications network (e.g., 3G, 4G, 5G, etc.), wireless local area network (e.g., a WiFi network), a Bluetooth™ network, and/or other network.

The one or more transceiver-like components (e.g., a wireless transceiver) of the communication component 135 may include an RF front end including one or more components, such as an amplifier, a mixer (also referred to as a signal multiplier) for signal down conversion, a frequency synthesizer (also referred to as an oscillator) that provides signals to the mixer, a baseband filter, an analog-to-digital converter (ADC), one or more power amplifiers, among other components. The RF front-end can generally handle selection and conversion of the wireless signals into a baseband or intermediate frequency and can convert the RF signals to the digital domain.

In some cases, a encoder-decoder (CODEC) may be implemented (e.g., by the processor 110) to encode and/or decode data transmitted and/or received using the one or more wireless transceivers. In some cases, encryption-decryption may be implemented (e.g., by the processor 110) to encrypt and/or decrypt data (e.g., according to the Advanced Encryption Standard (AES) and/or Data Encryption Standard (DES) standard) transmitted and/or received by the one or more wireless transceivers.

Device 100 may perform one or more processes described herein. Device 100 may perform these processes based on processor 110 executing software instructions stored by a non-transitory computer-readable medium, such as memory 115 and/or storage component 120. A computer-readable medium is defined herein as a non-transitory memory device. A memory device includes memory space within a single physical storage device or memory space spread across multiple physical storage devices.

Software instructions may be read into memory 115 and/or storage component 120 from another computer-readable medium or from another device via communication component 135. When executed, software instructions stored in memory 115 and/or storage component 120 may cause processor 110 to perform one or more processes described herein. Additionally, or alternatively, hardwired circuitry may be used in place of or in combination with software instructions to perform one or more processes described herein. Thus, aspects described herein are not limited to any specific combination of hardware circuitry and software.

The number and arrangement of components shown in FIG. 1 are provided as an example. In practice, device 100 may include additional components, fewer components, different components, or differently arranged components than those shown in FIG. 1. Additionally, or alternatively, a set of components (e.g., one or more components) of device 100 may perform one or more functions described as being performed by another set of components of device 100.

As previously mentioned, diabetes is a common metabolic disease that may lead to serious complications (e.g., cardiovascular disease, stroke, blindness, chronic renal failure, neuropathy, and even death). Diabetes is a growing global challenge, where one (1) in eleven (11) people presently suffer from diabetes worldwide, which is expected to nearly double within the next ten (10) years. When the blood glucose level (e.g., blood glucose concentration) in a body is uncontrolled, diabetes can lead to complications that may result in a lifetime of consequences for a patient. Therefore, regular monitoring and subsequent immediate control of the blood glucose level is needed.

Currently, some available methods for monitoring and control of blood glucose levels are based on enzyme reactions that require a painful puncturing procedure of a fingertip with a lance to extract the blood invasively. This procedure may make a patient unwilling to check their glucose level as frequently as required and may have a risk of wound infection. These invasive glucose monitoring methods may not provide continuous glucose monitoring for control.

Other current available methods for monitoring and control of the blood glucose level are based on determining the glucose level by measuring interstitial fluid glucose (IFG). However, the glucose concentration measurement obtained from IFG has a seven (7) to fifteen minute (15) lag in what glucose level the brain is experiencing and, thus, glucose concentration measurements obtained from IFG may be problematic for developing a closed loop control for an insulin pump. These methods measuring IFG require an invasive breaking of the skin barrier (by microneedles), changing the sensor frequently (often weekly), and calibration by a finger prick of up to twice a day.

To address the challenges of these invasive glucose monitoring methods, numerous efforts have been made towards developing alternative non-invasive methods of glucose detection comparable to the currently available invasive techniques. Some non-invasive methods for monitoring and control of the blood glucose level are based on the use of optical spectroscopy. However, for these optical methods the analysis of the received light signal is inherently complex because the glucose signal is typically very weak and easily interferes with other signals from a variety of molecules in the blood and tissue. The received light signal is dominated by glucose and water absorption and, as such, the spectroscopy sensors cannot differentiate between signals caused by glucose scattering and signals caused by water absorption. These optical methods can also be vulnerable to the variability and inhomogeneity of human skin that can continually change due to normal physiology (e.g., throughout the day). These methods also have no ability to isolate on an artery or vein to measure the blood glucose level. The optical methods require frequent calibration and multiple wavelengths need to be pulsed nearly simultaneously to capture readings without any background fluctuations. Therefore, improved systems and techniques for non-invasive glucose monitoring can be useful.

In one or more aspects, the systems and techniques employ photoacoustics for non-invasive glucose sensing. In one or more examples, the systems and techniques provide non-invasive glucose monitoring using photoacoustic ratiometric glucose (PARG) sensing that utilizes photoacoustic spectroscopy. The systems and techniques allow for wearable, calibration-free, continuous, non-invasive measurements of blood glucose concentration. Blood glucose measurements require a deeper penetration into the tissue than the methods that target IFG measurements to be able to obtain a glucose measurement that is instantaneous and represents the glucose level presently experienced by the brain. PARG allows for depth discrimination on an artery or vein to enable a blood glucose measurement.

Light absorption can be affected by glucose concentration, especially in the NIR region (e.g., including wavelengths from 780 nm to 2500 nm) of the electromagnetic spectrum. The correlation of glucose concentration with light absorption can be positive, negative, or minimal (e.g., approximately zero), depending upon the wavelength being used. PARG utilizes signals with three different wavelengths (λ₁, λ₂, and λ₃) to obtain a photoacoustic spectrum to determine blood glucose concentration. In one or more examples, a first wavelength (λ₁) may have a positive correlation with the blood glucose concentration, a second wavelength (λ₂) may have a negative correlation with the blood glucose concentration, and a third wavelength (λ₃) may have a minimal (e.g., approximately zero) correlation with the blood glucose concentration. In some examples, the first wavelength (λ₁) can have a first positive correlation with the blood glucose concentration, the second wavelength (λ₂) can have a second positive correlation with the blood glucose concentration, and the third wavelength (λ₃) can have a minimal (e.g., approximately zero) correlation with the blood glucose concentration, where the first positive correlation and the second positive correlation may be different positive correlations. In one or more examples, the first wavelength (λ₁) may have a first negative correlation with the blood glucose concentration, the second wavelength (λ₂) may have a second negative correlation with the blood glucose concentration, and the third wavelength (λ₃) may have a minimal (e.g., approximately zero) correlation with the blood glucose concentration, where the first negative correlation and the second negative correlation may have different negative correlations. The use of signals with three different wavelengths (λ₁, λ₂, and λ₃) may provide for the normalization of the glucose measurements as well as for a calibration-free approach. The use of signals with three different wavelengths (λ₁, λ₂, and λ₃) can also eliminate physiological features (e.g., blood volume change, body temperature, movement, etc.) other than glucose affecting the signals.

In one or more aspects, the systems and techniques can determine blood glucose concentration by analyzing a photoacoustic spectrum of the blood glucose concentration. For example, a photoacoustic system is provided that can obtain the photoacoustic spectrum of blood glucose concentration from in vivo living tissue (e.g., skin) of a patient. In the photoacoustic system, a transmitter (e.g., one or more VCSELs, for example in the form of a VCSEL array) can radiate signals (e.g., with three different wavelengths (λ₁, λ₂, and λ₃)) towards the living tissue to produce an acoustic response from the living tissue. In one or more examples, the signals can be transmitted (e.g., pulsed) sequentially within one or more (e.g., one, two, three, or etc.) microseconds or nanoseconds from one another.

In one or more examples, the signals can include a first signal with a first wavelength (λ₁), a second signal with a second wavelength (λ₂), and a third signal with a third wavelength (λ₃). In some cases, the first wavelength can have a first correlation with the blood glucose concentration, the second wavelength can have a second correlation with the blood glucose concentration, and the third wavelength can have a third correlation with the blood glucose concentration. In one or more examples, the first correlation may be a first positive correlation with the blood glucose concentration and the second correlation may be a first negative correlation with the blood glucose concentration, the first correlation may be a second positive correlation with the blood glucose concentration and the second correlation may be a third positive correlation with the blood glucose concentration, or the first correlation may be a second negative correlation with the blood glucose concentration and the second correlation may be a third negative correlation with the blood glucose concentration.

When the laser beam (e.g., radiated from the transmitter) irradiates the living tissue (e.g., skin), a thermal expansion within the tissue is produced, which generates an acoustic response in the form of an acoustic wave. The photoacoustic system can employ a receiver for receiving a response signal (having a photoacoustic spectrum) of the acoustic response. One or more processors (e.g., of the photoacoustic system) can determine a ratiometric analysis of the blood glucose concentration C_{gl} based on the received response signal. For instance, the one or more processors can determine a ratio (R) of characteristics of the response signal based on the first wavelength over characteristics of the response signal based on the second wavelength over characteristics of the response signal based on the third wavelength.

In one or more examples, the characteristics may be a photoacoustic intensity, a time integral (e.g., an area under the curve), or signal features (e.g., signal shape). For example, the one or more processors can determine a ratio (R) of a photoacoustic intensity of the response signal based on the first wavelength (e.g., ΔI_{OA}(λ₁)) over a photoacoustic intensity of the response signal based on the second wavelength (e.g., ΔI_{OA}(λ₂)) over a photoacoustic intensity of the response signal based on the third wavelength (e.g., ΔI_{OA}(λ₃)), such that the ratio R can be found by the following formula:

$$R = \frac{Δ {I}_{OA} ( {λ}_{1} ) / Δ {I}_{OA} ( {λ}_{2} )}{Δ {I}_{OA} ( {λ}_{3} )} \qquad ( equation 1 )$$

where I_{OA}(λ) represents the intensity (I) of optical absorption (OA) at a particular wavelength λ. In one or more examples, the blood glucose concentration can be determined based on the determined ratio R.

In some aspects, the radiometric analysis of the blood glucose concentration C_{gl} can be determined using the ratio R at three different wavelengths (λ₁, λ₂, and λ₃). In one or more examples, the blood glucose concentration can be determined by the following:

$${C}_{gl} = K \frac{R - {R}_{0}}{{R}_{gl} - R} \qquad ( equation 2 )$$

where R is the experimentally measured ratio of characteristics (e.g., I_{OA}) at three wavelengths (λ₁, λ₂, and λ₃), R₀ is the ratio at C_{gl}=0 (e.g., or approximately zero), R_{gl} corresponds to the ratio of characteristics (e.g., I_{OA}) of the known glucose spectrum at the chosen wavelengths (λ₁, λ₂, and λ₃), and K is a gain factor that can be defined with pre-calibration by fitting the equation to data obtained from a set of solutions with known glucose concentrations. In some examples, R₀ does not need to be zero (0) glucose concentration. For instance, in some examples, R₀ may be any low glucose level.

In some examples, the characteristics of the response signal based on the third wavelength (e.g., ΔI_{OA}(I₃)) may be used as a normalization factor for R. This normalization factor can account for physiological variations that affect the characteristics of the response signal based on the first wavelength (e.g., ΔI_{OA}(λ₁)) and the characteristics of the response signal based on the second wavelength (e.g., ΔI_{OA}(λ₂)) differently. For instance, the absorption of water may be different at the first wavelength (λ₁) than at the second wavelength (λ₂). For example, as the water content increases, ΔI_{OA}(λ₁)/ΔI_{OA}(λ₂) will decrease as a longer wavelength has a stronger water absorption. After normalization by ΔI_{OA}(λ₃), this effect can be canceled. Although the system is essentially calibration-free, an initial calibration of the system may be needed in some cases.

In some examples, the first signal, the second signal, and the third signal may be transmitted within ten microseconds or nanoseconds of each other. In one or more examples, the first wavelength, the second wavelength, and the third wavelength are each a NIR wavelength (e.g., a wavelength within 780 to 2500 nm, or a wavelength within 1000 to 2000 nm). In some examples, the blood glucose concentration is an absolute value (e.g., a quantity value). In one or more examples, the third correlation is between the first correlation and the second correlation. In some examples, the third correlation with the blood glucose concentration is approximately zero. In one or more examples, each transmitter of the one or more transmitters is a VCSEL.

FIG. 2 shows an example process for determining blood glucose concentration utilizing photoacoustics. In particular, FIG. 2 is a diagram illustrating an example process 200 utilizing photoacoustics for non-invasive glucose sensing. In FIG. 2, a signal 210 (e.g., a NIR signal) is shown to be transmitted towards red blood cells 205 (e.g., within an artery or vein in living tissue, such as within a finger 220) to produce an acoustic response. A response signal 215 (e.g., including a photoacoustic spectrum) produced from the acoustic response is shown.

In FIG. 2, also shown is an example system for determining blood glucose concentration utilizing photoacoustics. The system is shown to include a transmitter 225 (e.g., a laser diode light source) and a receiver 230 (e.g., an ultrasonic receiver). In one or more examples, the system may include one or more transmitters for transmitter 225. In some examples, a VCSEL may be employed for each transmitter of the one or more transmitters of the system.

During operation of the system of FIG. 2 for determining blood glucose concentration, the transmitter 225 may transmit three signals (e.g., a first signal, a second signal, and a third signal) towards living tissue (e.g., finger 220) to obtain blood glucose concentration within the living tissue. In one or more examples, the transmitter 225 (e.g., which may be in the form of one or more transmitters) may transmit each of the three signals (e.g., the first signal, the second signal, and the third signal) sequentially within a few microseconds or nanoseconds of each other. In some examples, the transmitter 225 may be in the form of an array of three transmitters (e.g., including transmitter 1, transmitter 2, and transmitter 3), where each transmitter may transmit a respective signal towards the living tissue within a few microseconds or nanoseconds of each other. For example, at time t₁, transmitter 1 may transmit the first signal towards the living tissue. At time t₂, which may be a few (e.g., 2, 3, 4, . . . 10, etc.) microseconds after time t₁, transmitter 2 may transmit the second signal towards the living tissue. At time t₃, which may be a few (e.g., 2, 3, 4, . . . 10, etc.) microseconds after time t₂, transmitter 3 may transmit the third signal towards the living tissue.

In one or more examples, the three signals may each have a different wavelength. For example, the first signal may have a first wavelength (λ₁), the second signal may have a second wavelength (λ₂), and the third signal may have a third wavelength (λ₃). In some examples, the first wavelength, the second wavelength, and the third wavelength are each a NIR wavelength. In one or more examples, the first wavelength (λ₁) has a first correlation with the blood glucose concentration, the second wavelength (λ₂) has a second correlation with the blood glucose concentration, and the third wavelength (λ₃) has a third correlation with the blood glucose concentration.

In some examples, the first correlation may be a positive correlation with the blood glucose concentration and the second correlation may be a negative correlation with the blood glucose concentration. In one or more examples, the first correlation may be a first positive correlation with the blood glucose concentration and the second correlation may be a second positive correlation with the blood glucose concentration, where the first positive correlation with the blood glucose concentration and the second positive correlation with the blood glucose concentration are different from each other. In one or more examples, the first correlation may be a first negative correlation with the blood glucose concentration and the second correlation may be a second negative correlation with the blood glucose concentration, where the first negative correlation with the blood glucose concentration and the second negative correlation with the blood glucose concentration are different from each other. In one or more examples, the third correlation may have a minimal correlation (e.g., approximately zero) with the blood glucose concentration. In some examples, the third correlation may be between the first correlation and the second correlation.

As the first signal, second signal, and third signal irradiate the living tissue (e.g., finger 220), a thermal expansion within the tissue (e.g., within the skin and/or artery) is produced, which generates an acoustic response in the form of an acoustic wave (e.g., response signals 235, 240, which are photoacoustic signals). The photoacoustic signal amplitude can be determined by:

$$A ( x ) = F ( x ) Γ ( x ) {μ}_{a} ( x ) \qquad ( equation 3 )$$

where F(x) is light fluence, F(x) is the Grüneisen coefficient, and μₐ(x) is a light absorption coefficient. F(x) is affected by both light absorption and scattering. In general, light absorption increases with glucose concentration, while light scattering decreases with glucose concentration. Γ(x) is proportional to (ν²β/Cᵥ), where ν is the speed of sound in tissue, β is the thermal coefficient of volume expansion of tissue, and Cᵥ is the specific heat capacity at a constant volume. ν, β, and (1/Cᵥ) all increase as the glucose concentration increases. The light absorption μₐ(x) is affected by the glucose concentration. A change in light absorption is caused by glucose bonding with water. Different wavelengths can have different correlations (e.g., in light absorption) with blood glucose concentration. For example, at some wavelengths, an increase in glucose concentration can lead to a stronger light absorption. Conversely, at other wavelengths, an increase in glucose concentration can cause a weaker light absorption. In one or more examples, it may be necessary to conduct in vitro and/or in vivo tests (e.g., for an initial calibration of the system) to determine the specific three wavelengths (e.g., λ₁, λ₂, and λ₃) to use for the system for ratiometric detection of blood glucose concentration.

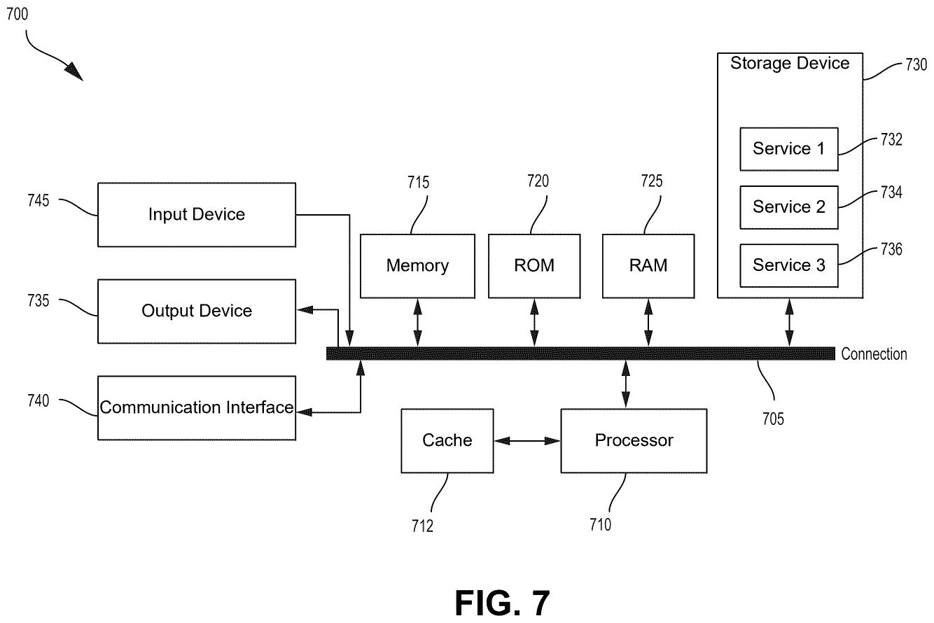

After an acoustic response is produced from the living tissue (e.g., finger 220), the receiver 230 can receive a response signal (e.g., response signals 235, 240) of the acoustic response. One or more processors (e.g., processor 110 of FIG. 1 and/or processor 710 of FIG. 7) can determine (e.g., by using equations 1 and 2) the blood glucose concentration C_{gl} within the living tissue (e.g., finger 220) based on a photoacoustic spectrum of the received response signal (e.g., response signals 235, 240). In one or more examples, the one or more processors may determine a ratio of characteristics of the response signal based on the first wavelength (λ₁) over characteristics of the response signal based on the second wavelength (λ₂) over characteristics of the response signal based on the third wavelength (λ₃). In one or more examples, the characteristics of the response signal may be a photoacoustic intensity, a time integral (e.g., an area under the curve), or signal features (e.g., signal shape). As such, when the characteristics of the response signal are a photoacoustic intensity (ΔI_{OA}), the ratio can be equal to the ratio R in equation 1. In one or more examples, the characteristics of the response signal based on the third wavelength include a normalization factor. The one or more processors of the system can determine the blood glucose concentration in the living tissue (e.g., 220) based on the determined ratio. In one or more examples, the blood glucose concentration is an absolute value (e.g., a quantitative value).

FIG. 2 also shows a graph 270 illustrating depth discrimination of the living tissue (e.g., finger 220) over time. In the graph 270, the x-axis represents time in microseconds (s), and the y-axis represents amplitude in arbitrary units (a.u.). In some cases, the amplitude may be measured in volts (v). The graph 270 shows a plot of the received response signal (e.g., photoacoustic spectrum) from the tissue (e.g., finger 220). PAPG can obtain individual signal characteristics from different depths of living tissue and, as such, can perform depth discrimination of the living tissue. Skin and arteries reside at different depths of the living tissue and have unique signal characteristics. Simple time of flight (ToF) range gating can be used to separate out the arteries containing the blood.

As shown in the graph 270, an electromagnetic interference (EMI) pulse 245 is shown at the start of the time. This EMI pulse 245 is produced by the transmission of the three signals (e.g., the first signal, the second signal, and the third signal) by the transmitter 225. After the EMI pulse 245, a skin signal 250 is shown on the graph 270. This skin signal 250 is produced by the three signals traveling to a depth of the skin of the finger 220. After the skin signal 250, an artery signal 255 is shown. This artery signal 255 is produced by the three signals traveling to a depth of an artery of the finger 220. After the artery signal 255, an echo EMI 260 is shown on the graph 270. The echo EMI 260 is produced by the three signals traveling to a depth of blood within the finger 220.

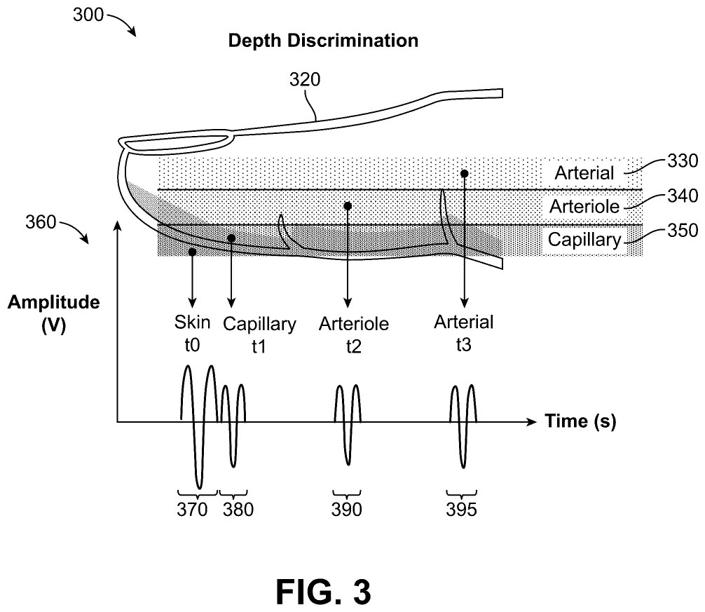

FIG. 3 is a diagram illustrating an example 300 of depth discrimination by using photoacoustics. In FIG. 3, depth discrimination of a living tissue (e.g., in the form of a finger 320) using photoacoustics is shown. FIG. 3 shows a graph 360 illustrating depth discrimination of the living tissue (e.g., finger 320) over time. As mentioned, PAPG can obtain individual signal characteristics from different depths of living tissue and, thus, can perform depth discrimination of the living tissue. Arteries, veins, and capillaries reside at different depths of the living tissue and have unique signal characteristics. ToF range gating may be used to separate out these vessels.

In the graph 360, the x-axis represents time in seconds (s), and the y-axis represents amplitude in volts (v). The graph 360 shows a plot of a received response signal (e.g., including a photoacoustic spectrum) from the living tissue (e.g., finger 320). As shown in the graph 360, at time to, a skin signal 370 is shown. This skin signal 370 is produced by the transmission of three signals (e.g., first signal, second signal, and third signal, each at a different wavelength) traveling to a depth of the skin of the finger 320. At time t₁, a capillary signal 380 is shown. This capillary signal 380 is produced by the transmission of the three signals traveling to a depth of the capillary tissue 350 of the finger 320. At time t₂, a arteriole signal 390 is shown. This arteriole signal 390 is produced by the transmission of the three signals traveling to a depth of the arteriole tissue 340 of the finger 320. At time t₃, an arterial signal 395 is shown. This arterial signal 395 is produced by the transmission of the three signals traveling to a depth of the arterial tissue 330 of the finger 320.

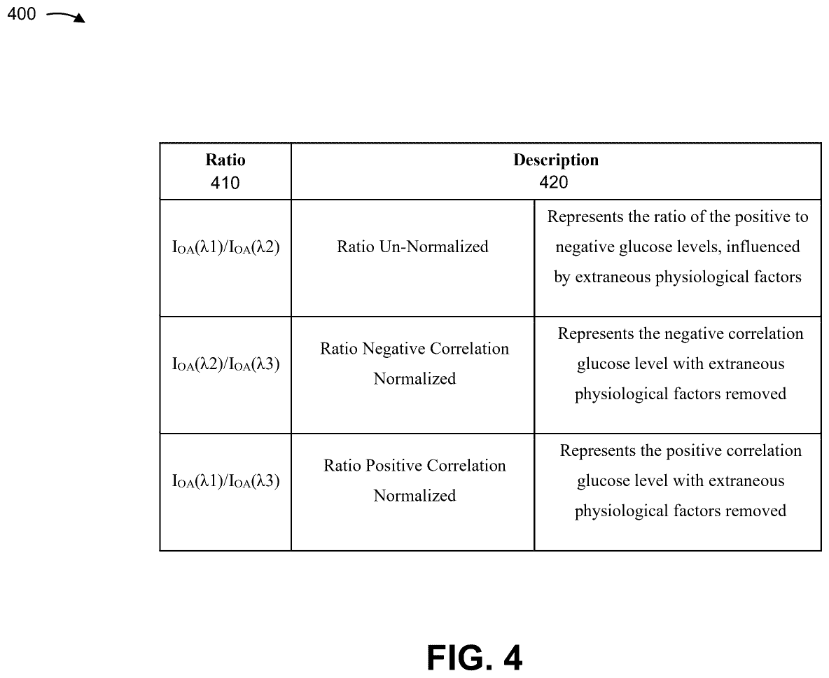

FIG. 4 is a table 400 illustrating different ratios of photoacoustic intensities of a response signal with a photoacoustic spectrum based on different wavelengths, where the ratios may be employed when utilizing photoacoustics for non-invasive glucose sensing. In the table 400 of FIG. 4, a ratio 410 column and a description 420 column are shown. In the ratio 410 column, different ratios of photoacoustic intensities are shown. For example, in the first row of the table 400, in the ratio 410 column, a ratio of a photoacoustic intensity of the response signal based on the first wavelength (ΔI_{OA}(λ₁)) over a photoacoustic intensity of the response signal based on the second wavelength (ΔI_{OA}(λ₂)) is shown. As shown in the description 420 column, this ratio is un-normalized and represents the ratio of the positive to negative glucose levels, influenced by extraneous physiological factors. In the second row of the table 400, in the ratio 410 column, a ratio of a photoacoustic intensity of the response signal based on the second wavelength (ΔI_{OA}(λ₂)) over a photoacoustic intensity of the response signal based on the third wavelength (ΔI_{OA}(λ₃)) is shown. As shown in the description 420 column, this ratio is normalized, has a negative correlation with the glucose concentration, and represents a negative correlation with the glucose level with extraneous physiological factors removed. In the third row of the table 400, in the ratio 410 column, a ratio of a photoacoustic intensity of the response signal based on the first wavelength (ΔI_{OA}(λ₁)) over a photoacoustic intensity of the response signal based on the third wavelength (ΔI_{OA}(λ₃)) is shown. As shown in the description 420 column, this ratio is normalized, has a positive correlation with the glucose concentration, and represents a positive correlation with the glucose level with extraneous physiological factors removed.

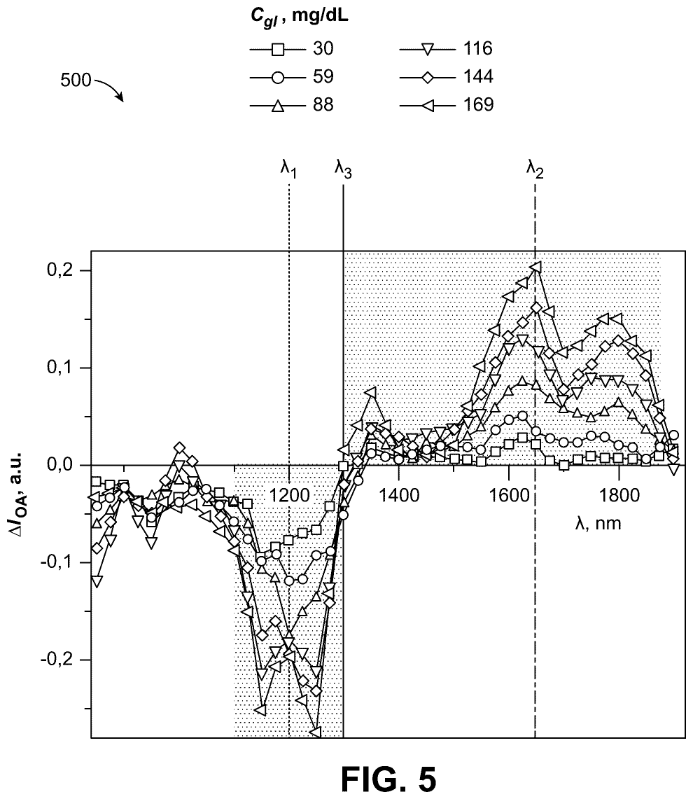

FIG. 5 is a graph 500 illustrating an example of a photoacoustic spectra of various different glucose concentrations. In the graph 500 of FIG. 5, the x-axis represents wavelengths (λ) in nanometers (nm), and the y-axis represents a change in (or delta) photoacoustic (PA) intensity (ΔI_{OA}, represented in arbitrary units (a.u.)) of response signals (e.g., each including a photoacoustic spectrum). The delta PA intensity ΔI_{OA} is the change in PA intensity for the different concentrations versus 0 mg/dL. The graph 500 shows a respective photoacoustic spectrum for each glucose concentration of the different glucose concentrations plotted over a 850 to 1900 nm spectral range. For example, the graph 500 shows photoacoustic spectra for a total of six (6) different glucose concentrations C_{gl}, in milligrams per deciliter (mg/dL). In the graph 500, the photoacoustic spectra are shown to have positive and negative photoacoustic intensity (ΔI_{OA}) changes across the different wavelengths. For example, a negative region is shown to fall within the 1150 to 1250 nm range, a positive region is shown to fall within the 1550 to 1800 nm range, and an approximately zero region is shown to fall around 1300 nm.

In one or more examples, the different wavelengths (e.g., λ₁, λ₂, and λ₃) for the first transmit signal, the second transmit signal, and the third transmit signal, respectively, may be chosen by observing the graph 500 (e.g., or a similar graph). For example, a first wavelength (λ₁) for the first transmit signal may be chosen to be within the 1150 to 1250 nm range (e.g., 1200 nm) such that the first wavelength has a negative correlation with the glucose concentration. A second wavelength (λ₂) for the second transmit signal may be chosen to be within the 1550 to 1800 nm range (e.g., 1650 nm) such that the second wavelength has a positive correlation with the glucose concentration. A third wavelength (λ₃) for the third transmit signal may be chosen to be around 1300 nm such that the third wavelength has a minimal (e.g., approximately zero) correlation with the glucose concentration.

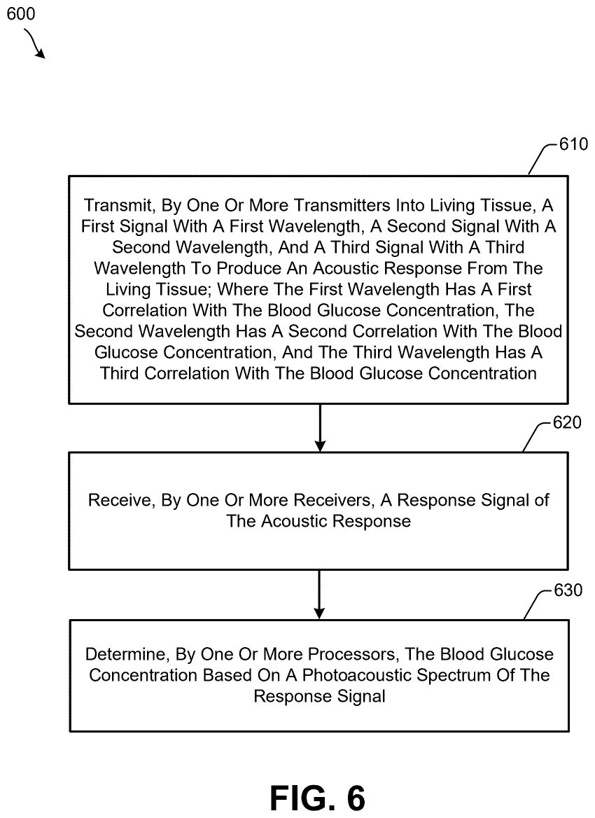

FIG. 6 is a flow chart illustrating an example of a process 600 for to determining a blood glucose concentration. The process 600 can be performed by a computing device (e.g., device 100 of FIG. 1, a transmitter such as the transmitter 225 of FIG. 2, a receiver such as the receiver 230 of FIG. 2, and/or a transducer, which may include the transmitter 225 and the receiver 230 of FIG. 2, a computing device or computing system 700 of FIG. 7, any combination thereof, and/or other device) or by a component or system (e.g., a chipset, one or more processors such as one or more central processing units (CPUs), digital signal processors (DSPs), graphics processing units (GPUs), any combination thereof, and/or other type of processor(s), or other component or system) of the computing device. The operations of the process 600 may be implemented as software components that are executed and run on one or more processors (e.g., processor 710 of FIG. 7 or other processor(s)). Further, the transmission and reception of signals by the computing device in the process 600 may be enabled, for example, by one or more antennas and/or one or more receivers, transmitters (e.g., a vertical-cavity surface-emitting laser (VCSEL) or an array of VCSELs), and/or transceivers (e.g., wireless transceiver(s)).

At block 610, the computing device (or component thereof, such as at least one transmitter or transceiver) can transmit, into living tissue, a first signal with a first wavelength, a second signal with a second wavelength, and a third signal with a third wavelength to produce an acoustic response from the living tissue. The first wavelength has a first correlation with the blood glucose concentration, the second wavelength has a second correlation with the blood glucose concentration, and the third wavelength has a third correlation with the blood glucose concentration. In some aspects, the third correlation is between the first correlation and the second correlation. In some cases, the third correlation is a minimal correlation (e.g., a correlation value close to zero) with the blood glucose concentration.

For example, in some cases, the first correlation is a first positive correlation with the blood glucose concentration and the second correlation is a first negative correlation with the blood glucose concentration. In some cases, the first correlation is a second positive correlation with the blood glucose concentration and the second correlation is a third positive correlation with the blood glucose concentration. In some cases, the first correlation is a second negative correlation with the blood glucose concentration and the second correlation is a third negative correlation with the blood glucose concentration. In some examples, the first positive correlation, the second positive correlation, and the third positive correlation are different positive correlations. In some examples, the first negative correlation, the second negative correlation, and the third negative correlation are different negative correlations.

In some aspects, the first signal, the second signal, and the third signal are transmitted within a certain time duration of each other (e.g., within ten microseconds of each other). In some examples, the first wavelength, the second wavelength, and the third wavelength are each a near infrared (NIR) wavelength.

At block 620, the computing device (or component thereof, such as at least one receiver or transceiver) can receive a response signal of the acoustic response.

At block 630, the computing device (or component thereof, such as at least one processor) can determine the blood glucose concentration based on a photoacoustic spectrum of the response signal. In some cases, the blood glucose concentration is an absolute value. In some aspects, to determine the blood glucose concentration based on a photoacoustic spectrum of the response signal, the computing device (or component thereof, such as at least one processor) can determine a ratio of characteristics of the response signal based on the first wavelength over characteristics of the response signal based on the second wavelength over characteristics of the response signal based on the third wavelength. In some cases, the characteristics can be a photoacoustic intensity, a time integral, signal features, and/or other characteristics. For example, the one or more processors can determine a ratio (R) of a photoacoustic intensity of the response signal based on the first wavelength (e.g., ΔI_{OA}(λ₁)) over a photoacoustic intensity of the response signal based on the second wavelength (e.g., ΔI_{OA}(λ₂)) over a photoacoustic intensity of the response signal based on the third wavelength (e.g., ΔI_{OA}(λ₃)). In one illustrative example, the ratio R can be found by the following formula:

$$R = \frac{Δ {I}_{OA} ( {λ}_{1} ) / Δ {I}_{OA} ( {λ}_{2} )}{Δ {I}_{OA} ( {λ}_{3} )} \qquad ( equation 1 )$$

In some cases, the characteristics of the response signal based on the third wavelength (e.g., ΔI_{OA}(λ₃)) can include or be used as a normalization factor. In some aspects, the computing device (or component thereof, such as at least one processor) can determine the blood glucose concentration based on the ratio.

In some cases, the computing device of process 600 may include various components, such as one or more input devices, one or more output devices, one or more processors, one or more microprocessors, one or more microcomputers, one or more cameras, one or more sensors, and/or other component(s) that are configured to carry out the steps of processes described herein. In some examples, the computing device may include a display, one or more network interfaces configured to communicate and/or receive the data, any combination thereof, and/or other component(s). The one or more network interfaces may be configured to communicate and/or receive wired and/or wireless data, including data according to the 3G, 4G, 5G, and/or other cellular standard, data according to the Wi-Fi (802.11x) standards, data according to the Bluetooth™ standard, data according to the Internet Protocol (IP) standard, and/or other types of data.

The components of the computing device of process 600 can be implemented in circuitry. For example, the components can include and/or can be implemented using electronic circuits or other electronic hardware, which can include one or more programmable electronic circuits (e.g., microprocessors, graphics processing units (GPUs), digital signal processors (DSPs), central processing units (CPUs), and/or other suitable electronic circuits), and/or can include and/or be implemented using computer software, firmware, or any combination thereof, to perform the various operations described herein. The computing device may further include a display (as an example of the output device or in addition to the output device), a network interface configured to communicate and/or receive the data, any combination thereof, and/or other component(s). The network interface may be configured to communicate and/or receive Internet Protocol (IP) based data or other type of data.

The process 600 is illustrated as a logical flow diagram, the operations of which represent a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, process 600 may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable or machine-readable storage medium, for example, in the form of a computer program comprising a plurality of instructions executable by one or more processors. The computer-readable or machine-readable storage medium may be non-transitory.

FIG. 7 is a block diagram illustrating an example of a computing system 700, which may be employed for utilizing photoacoustics for non-invasive glucose sensing. In particular, FIG. 7 illustrates an example of computing system 700, which can be for example any computing device making up internal computing system, a remote computing system, a camera, or any component thereof in which the components of the system are in communication with each other using connection 705. Connection 705 can be a physical connection using a bus, or a direct connection into processor 710, such as in a chipset architecture. Connection 705 can also be a virtual connection, networked connection, or logical connection.

In some aspects, computing system 700 is a distributed system in which the functions described in this disclosure can be distributed within a datacenter, multiple data centers, a peer network, etc. In some aspects, one or more of the described system components represents many such components each performing some or all of the function for which the component is described. In some aspects, the components can be physical or virtual devices.

Example system 700 includes at least one processing unit (CPU or processor) 710 and connection 705 that communicatively couples various system components including system memory 715, such as read-only memory (ROM) 720 and random access memory (RAM) 725 to processor 710. Computing system 700 can include a cache 712 of high-speed memory connected directly with, in close proximity to, or integrated as part of processor 710.

Processor 710 can include any general purpose processor and a hardware service or software service, such as services 732, 734, and 736 stored in storage device 730, configured to control processor 710 as well as a special-purpose processor where software instructions are incorporated into the actual processor design. Processor 710 may essentially be a completely self-contained computing system, containing multiple cores or processors, a bus, memory controller, cache, etc. A multi-core processor may be symmetric or asymmetric.

To enable user interaction, computing system 700 includes an input device 745, which can represent any number of input mechanisms, such as a microphone for speech, a touch-sensitive screen for gesture or graphical input, keyboard, mouse, motion input, speech, etc. Computing system 700 can also include output device 735, which can be one or more of a number of output mechanisms. In some instances, multimodal systems can enable a user to provide multiple types of input/output to communicate with computing system 700.

Computing system 700 can include communications interface 740, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple™ Lightning™ port/plug, an Ethernet port/plug, a fiber optic port/plug, a proprietary wired port/plug, 3G, 4G, 5G and/or other cellular data network wireless signal transfer, a Bluetooth™ wireless signal transfer, a Bluetooth™ low energy (BLE) wireless signal transfer, an IBEACON™ wireless signal transfer, a radio-frequency identification (RFID) wireless signal transfer, near-field communications (NFC) wireless signal transfer, dedicated short range communication (DSRC) wireless signal transfer, 802.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof.

The communications interface 740 may also include one or more range sensors (e.g., LIDAR sensors, laser range finders, RF radars, ultrasonic sensors, and infrared (IR) sensors) configured to collect data and provide measurements to processor 710, whereby processor 710 can be configured to perform determinations and calculations needed to obtain various measurements for the one or more range sensors. In some examples, the measurements can include time of flight, wavelengths, azimuth angle, elevation angle, range, linear velocity and/or angular velocity, or any combination thereof. The communications interface 740 may also include one or more Global Navigation Satellite System (GNSS) receivers or transceivers that are used to determine a location of the computing system 700 based on receipt of one or more signals from one or more satellites associated with one or more GNSS systems. GNSS systems include, but are not limited to, the US-based GPS, the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 730 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically erasable programmable read-only memory (EEPROM), flash EPROM (FLASHEPROM), cache memory (e.g., Level 1 (L1) cache, Level 2 (L2) cache, Level 3 (L3) cache, Level 4 (L4) cache, Level 5 (L5) cache, or other (L #) cache), resistive random-access memory (RRAM/ReRAM), phase change memory (PCM), spin transfer torque RAM (STT-RAM), another memory chip or cartridge, and/or a combination thereof.

The storage device 730 can include software services, servers, services, etc., that when the code that defines such software is executed by the processor 710, it causes the system to perform a function. In some aspects, a hardware service that performs a particular function can include the software component stored in a computer-readable medium in connection with the necessary hardware components, such as processor 710, connection 705, output device 735, etc., to carry out the function. The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Specific details are provided in the description above to provide a thorough understanding of the aspects and examples provided herein, but those skilled in the art will recognize that the application is not limited thereto. Thus, while illustrative aspects of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, aspects can be utilized in any number of environments and applications beyond those described herein without departing from the broader scope of the specification. The specification and drawings are, accordingly, to be regarded as illustrative rather than restrictive. For the purposes of illustration, methods were described in a particular order. It should be appreciated that in alternate aspects, the methods may be performed in a different order than that described.

For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the aspects in unnecessary detail. In other instances, well-known circuits, processes, algorithms, structures, and techniques may be shown without unnecessary detail in order to avoid obscuring the aspects.

Further, those of skill in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Individual aspects may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

In some aspects the computer-readable storage devices, mediums, and memories can include a cable or wireless signal containing a bitstream and the like. However, when mentioned, non-transitory computer-readable storage media expressly exclude media such as energy, carrier signals, electromagnetic waves, and signals per se.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof, in some cases depending in part on the particular application, in part on the desired design, in part on the corresponding technology, etc.

The various illustrative logical blocks, modules, and circuits described in connection with the aspects disclosed herein may be implemented or performed using hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general-purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” or “communicatively coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, A and B and C, or any duplicate information or data (e.g., A and A, B and B, C and C, A and A and B, and so on), or any other ordering, duplication, or combination of A, B, and C. The language “at least one of” a set and/or “one or more” of a set does not limit the set to the items listed in the set. For example, claim language reciting “at least one of A and B” or “at least one of A or B” may mean A, B, or A and B, and may additionally include items not listed in the set of A and B. The phrases “at least one” and “one or more” are used interchangeably herein.

Claim language or other language reciting “at least one processor configured to,” “at least one processor being configured to,” “one or more processors configured to,” “one or more processors being configured to,” or the like indicates that one processor or multiple processors (in any combination) can perform the associated operation(s). For example, claim language reciting “at least one processor configured to: X, Y, and Z” means a single processor can be used to perform operations X, Y, and Z; or that multiple processors are each tasked with a certain subset of operations X, Y, and Z such that together the multiple processors perform X, Y, and Z; or that a group of multiple processors work together to perform operations X, Y, and Z. In another example, claim language reciting “at least one processor configured to: X, Y, and Z” can mean that any single processor may only perform at least a subset of operations X, Y, and Z.

Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions.

Where reference is made to an entity (e.g., any entity or device described herein) performing functions or being configured to perform functions (e.g., steps of a method), the entity may be configured to cause one or more elements (individually or collectively) to perform the functions. The one or more components of the entity may include at least one memory, at least one processor, at least one communication interface, another component configured to perform one or more (or all) of the functions, and/or any combination thereof. Where reference to the entity performing functions, the entity may be configured to cause one component to perform all functions, or to cause more than one component to collectively perform the functions. When the entity is configured to cause more than one component to collectively perform the functions, each function need not be performed by each of those components (e.g., different functions may be performed by different components) and/or each function need not be performed in whole by only one component (e.g., different components may perform different sub-functions of a function).

The various illustrative logical blocks, modules, engines, circuits, and algorithm steps described in connection with the embodiments disclosed herein may be implemented as electronic hardware, computer software, firmware, or combinations thereof. To clearly illustrate this interchangeability of hardware and software, various illustrative components, blocks, engines, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as engines, modules, or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for encoding and decoding, or incorporated in a combined encoder-decoder (CODEC).

Illustrative aspects of the disclosure include:

Aspect 1. An apparatus configured to determine a blood glucose concentration, the apparatus comprising: at least one transmitter configured to transmit, into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; at least one receiver configured to receive a response signal of the acoustic response; and at least one processor configured to determine the blood glucose concentration based on a photoacoustic spectrum of the response signal.

Aspect 2. The apparatus of Aspect 1, wherein one of the first correlation is a first positive correlation with the blood glucose concentration and the second correlation is a first negative correlation with the blood glucose concentration, the first correlation is a second positive correlation with the blood glucose concentration and the second correlation is a third positive correlation with the blood glucose concentration, or the first correlation is a second negative correlation with the blood glucose concentration and the second correlation is a third negative correlation with the blood glucose concentration.

Aspect 3. The apparatus of Aspect 2, the first positive correlation, the second positive correlation, and the third positive correlation are different positive correlations.

Aspect 4. The apparatus of any of Aspects 2 or 3, wherein the first negative correlation, the second negative correlation, and the third negative correlation are different negative correlations.

Aspect 5. The apparatus of any of Aspects 1 to 4, wherein, to determine the blood glucose concentration based on a photoacoustic spectrum of the response signal, the at least one processor is configured to: determine a ratio of characteristics of the response signal based on the first wavelength over characteristics of the response signal based on the second wavelength over characteristics of the response signal based on the third wavelength.

Aspect 6. The apparatus of Aspect 5, wherein the characteristics of the response signal are one of a photoacoustic intensity, a time integral, or signal features.

Aspect 7. The apparatus of any of Aspects 5 or 6, wherein the characteristics of the response signal based on the third wavelength include a normalization factor.

Aspect 8. The apparatus of any of Aspects 5 to 7, wherein the at least one processor is configured to determine the blood glucose concentration based on the ratio.

Aspect 9. The apparatus of any of Aspects 1 to 8, wherein the first signal, the second signal, and the third signal are transmitted within ten microseconds of each other.

Aspect 10. The apparatus of any of Aspects 1 to 9, wherein the first wavelength, the second wavelength, and the third wavelength are each a near infrared (NIR) wavelength.

Aspect 11. The apparatus of any of Aspects 1 to 10, wherein the blood glucose concentration is an absolute value.

Aspect 12. The apparatus of any of Aspects 1 to 11, wherein the third correlation is between the first correlation and the second correlation.

Aspect 13. The apparatus of any of Aspects 1 to 12, wherein the third correlation is a minimal correlation with the blood glucose concentration.

Aspect 14. The apparatus of any of Aspects 1 to 13, wherein each transmitter of the at least one transmitter is a vertical-cavity surface-emitting laser (VCSEL).

Aspect 15. The apparatus of any of Aspects 1 to 14, further comprising at least one memory coupled to the at least one processor, the at least one memory configured to store information associated with the photoacoustic spectrum of the response signal.

Aspect 16. A method for determining a blood glucose concentration, the method comprising: transmitting, by at least one transmitter into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration; receiving, by at least one receiver, a response signal of the acoustic response; and determining, by at least one processor, the blood glucose concentration based on a photoacoustic spectrum of the response signal.

Aspect 17. The method of Aspect 16, wherein one of the first correlation is a first positive correlation with the blood glucose concentration and the second correlation is a first negative correlation with the blood glucose concentration, the first correlation is a second positive correlation with the blood glucose concentration and the second correlation is a third positive correlation with the blood glucose concentration, or the first correlation is a second negative correlation with the blood glucose concentration and the second correlation is a third negative correlation with the blood glucose concentration.

Aspect 18. The method of Aspect 17, the first positive correlation, the second positive correlation, and the third positive correlation are different positive correlations.

Aspect 19. The method of any of Aspects 17 or 18, wherein the first negative correlation, the second negative correlation, and the third negative correlation are different negative correlations.

Aspect 20. The method of any of Aspects 16 to 19, wherein determining, by the at least one processor, the blood glucose concentration based on a photoacoustic spectrum of the response signal comprises: determining, by the at least one processor, a ratio of characteristics of the response signal based on the first wavelength over characteristics of the response signal based on the second wavelength over characteristics of the response signal based on the third wavelength.

Aspect 21. The method of Aspect 20, wherein the characteristics of the response signal are one of a photoacoustic intensity, a time integral, or signal features.

Aspect 22. The method of any of Aspects 20 or 21, wherein the characteristics of the response signal based on the third wavelength include a normalization factor.

Aspect 23. The method of any of Aspects 20 to 22, wherein the blood glucose concentration is determined based on the ratio.

Aspect 24. The method of any of Aspects 16 to 23, wherein the first signal, the second signal, and the third signal are transmitted within ten microseconds of each other.

Aspect 25. The method of any of Aspects 16 to 24, wherein the first wavelength, the second wavelength, and the third wavelength are each a near infrared (NIR) wavelength.

Aspect 26. The method of any of Aspects 16 to 25, wherein the blood glucose concentration is an absolute value.

Aspect 27. The method of any of Aspects 16 to 26, wherein the third correlation is between the first correlation and the second correlation.

Aspect 28. The method of any of Aspects 16 to 27, wherein the third correlation is a minimal correlation with the blood glucose concentration.

Aspect 29. The method of any of Aspects 16 to 28, wherein each transmitter of the at least one transmitter is a vertical-cavity surface-emitting laser (VCSEL).

Aspect 30. A non-transitory computer-readable medium having stored thereon instructions that, when executed by one or more processors, cause the one or more processors to perform operations according to any of Aspects 16 to 29.

Aspect 31. An apparatus including one or more means for performing operations according to any of Aspects 16 to 29.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.”

What is claimed is:

1. An apparatus configured to determine a blood glucose concentration, the apparatus comprising:
at least one transmitter configured to transmit, into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration;
at least one receiver configured to receive a response signal of the acoustic response; and
at least one processor configured to determine the blood glucose concentration based on a photoacoustic spectrum of the response signal.

2. The apparatus of claim 1, wherein one of the first correlation is a first positive correlation with the blood glucose concentration and the second correlation is a first negative correlation with the blood glucose concentration, the first correlation is a second positive correlation with the blood glucose concentration and the second correlation is a third positive correlation with the blood glucose concentration, or the first correlation is a second negative correlation with the blood glucose concentration and the second correlation is a third negative correlation with the blood glucose concentration.

3. The apparatus of claim 2, the first positive correlation, the second positive correlation, and the third positive correlation are different positive correlations.

4. The apparatus of claim 2, wherein the first negative correlation, the second negative correlation, and the third negative correlation are different negative correlations.

5. The apparatus of claim 1, wherein, to determine the blood glucose concentration based on a photoacoustic spectrum of the response signal, the at least one processor is configured to:
determine a ratio of characteristics of the response signal based on the first wavelength over characteristics of the response signal based on the second wavelength over characteristics of the response signal based on the third wavelength.

6. The apparatus of claim 5, wherein the characteristics of the response signal are one of a photoacoustic intensity, a time integral, or signal features.

7. The apparatus of claim 5, wherein the characteristics of the response signal based on the third wavelength include a normalization factor.

8. The apparatus of claim 5, wherein the at least one processor is configured to determine the blood glucose concentration based on the ratio.

9. The apparatus of claim 1, wherein the first signal, the second signal, and the third signal are transmitted within ten microseconds of each other.

10. The apparatus of claim 1, wherein the first wavelength, the second wavelength, and the third wavelength are each a near infrared (NIR) wavelength.

11. The apparatus of claim 1, wherein the blood glucose concentration is an absolute value.

12. The apparatus of claim 1, wherein the third correlation is between the first correlation and the second correlation.

13. The apparatus of claim 1, wherein the third correlation is a minimal correlation with the blood glucose concentration.

14. The apparatus of claim 1, wherein each transmitter of the at least one transmitter is a vertical-cavity surface-emitting laser (VCSEL).

15. The apparatus of claim 1, further comprising at least one memory coupled to the at least one processor, the at least one memory configured to store information associated with the photoacoustic spectrum of the response signal.

16. A method for determining a blood glucose concentration, the method comprising:
transmitting, by at least one transmitter into living tissue, a first signal with a first wavelength, a second signal with a second wavelength, and a third signal with a third wavelength to produce an acoustic response from the living tissue, wherein the first wavelength has a first correlation with the blood glucose concentration, the second wavelength has a second correlation with the blood glucose concentration, and the third wavelength has a third correlation with the blood glucose concentration;
receiving, by at least one receiver, a response signal of the acoustic response; and
determining, by at least one processor, the blood glucose concentration based on a photoacoustic spectrum of the response signal.

17. The method of claim 16, wherein one of the first correlation is a first positive correlation with the blood glucose concentration and the second correlation is a first negative correlation with the blood glucose concentration, the first correlation is a second positive correlation with the blood glucose concentration and the second correlation is a third positive correlation with the blood glucose concentration, or the first correlation is a second negative correlation with the blood glucose concentration and the second correlation is a third negative correlation with the blood glucose concentration.

18. The method of claim 17, the first positive correlation, the second positive correlation, and the third positive correlation are different positive correlations.

19. The method of claim 17, wherein the first negative correlation, the second negative correlation, and the third negative correlation are different negative correlations.

20. The method of claim 16, wherein determining, by the at least one processor, the blood glucose concentration based on a photoacoustic spectrum of the response signal comprises:
determining, by the at least one processor, a ratio of characteristics of the response signal based on the first wavelength over characteristics of the response signal based on the second wavelength over characteristics of the response signal based on the third wavelength.

21. The method of claim 20, wherein the characteristics of the response signal are one of a photoacoustic intensity, a time integral, or signal features.

22. The method of claim 20, wherein the characteristics of the response signal based on the third wavelength is include normalization factor.

23. The method of claim 20, wherein the blood glucose concentration is determined based on the ratio.

24. The method of claim 16, wherein the first signal, the second signal, and the third signal are transmitted within ten microseconds of each other.

25. The method of claim 16, wherein the first wavelength, the second wavelength, and the third wavelength are each a near infrared (NIR) wavelength.

26. The method of claim 16, wherein the blood glucose concentration is an absolute value.

27. The method of claim 16, wherein the third correlation is between the first correlation and the second correlation.

28. The method of claim 16, wherein the third correlation is a minimal correlation with the blood glucose concentration.

29. The method of claim 16, wherein each transmitter of the at least one transmitter is a vertical-cavity surface-emitting laser (VCSEL).
